(19) United States
(12) Patent Application Publication
(10) Pub. No.: US 2021/0228187 A1
(43) Pub. Date: Jul. 29, 2021

(54) SYSTEM AND METHODS FOR CONTRAST-ENHANCED ULTRASOUND IMAGING

(71) Applicant: GE Precision Healthcare LLC, Milwaukee, WI (US)

(72) Inventor: Yelena Viktorovna Tsymbalenko, Mequon, WI (US)

(21) Appl. No.: 16/776,147

(22) Filed: Jan. 29, 2020

(51) Int. Cl. A61B 8/08; A61B 8/00; G06T 7/00

(52) U.S. Cl. CPC A61B 8/481; A61B 8/469; G06T 2207/20084; G06T 2207/10132; G06T 2207/30004; G06T 7/0012

(57) ABSTRACT

Methods and systems are provided for automatically characterizing contrast agent microbubbles in contrast-enhanced ultrasound images. In one example, a method includes generating, via a contrast bubble model, a density map of contrast agent microbubbles in a region of interest (ROI) of a contrast-enhanced ultrasound image and displaying the density map on a display device.

### TECHNICAL FIELD

Embodiments of the subject matter disclosed herein relate to ultrasound imaging, and more particularly, to contrast-enhanced ultrasound imaging.

### BACKGROUND

Medical ultrasound is an imaging modality that employs ultrasound waves to probe the internal structures of a body of a patient and produce a corresponding image. For example, an ultrasound probe comprising a plurality of transducer elements emits ultrasonic pulses which reflect or echo, refract, or are absorbed by structures in the body. The ultrasound probe then receives reflected echoes, which are processed into an image. Ultrasound images of the internal structures may be saved for later analysis by a clinician to aid in diagnosis and/or displayed on a display device in real time or near real time.

### SUMMARY

In one embodiment, a method includes generating, via a contrast bubble model, a density map of contrast agent microbubbles in a region of interest (ROI) of a contrast-enhanced ultrasound image and displaying the density map on a display device.

The above advantages and other advantages, and features of the present description will be readily apparent from the following Detailed Description when taken alone or in connection with the accompanying drawings. It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

Various aspects of this disclosure may be better understood upon reading the following detailed description and upon reference to the drawings in which:

FIG. 1 shows a block diagram of an ultrasound system, according to an embodiment;

FIG. 2 is a schematic diagram illustrating a system for automatic contrast agent bubble characterization, according to an embodiment;

FIG. 3 is a flow chart illustrating a method for automatically characterizing contrast agent microbubbles in a contrast-enhanced ultrasound image, according to an embodiment; and

FIGS. 4 and 5 show example graphical user interfaces including contrast agent microbubble density maps generated according to the method of FIG. 3.

FIG. 6 shows an example graphical user interface including a plot of contrast agent microbubble counts in a region of interest over time.

### DETAILED DESCRIPTION

Ultrasound images acquired during a medical ultrasound exam may be used to diagnose a patient condition, which may include one or more clinicians analyzing the ultrasound images for abnormalities, measuring certain anatomical features imaged in the ultrasound images, and so forth. Some ultrasound imaging procedures, referred to as contrast-enhanced ultrasound imaging, include the administration of a contrast agent to a patient and subsequent imaging of certain anatomical features, such as the carotid artery. The contrast agent used in contrast-enhanced ultrasound may include microbubbles (approximately 1-8 μm) filled with a low solubility gas such as perfluorinated gas, and stabilized with a phospholipid or protein shell. The contrast agent microbubbles generate a non-linear response when subject to ultrasonic signals from an ultrasound probe, resulting in multiple harmonics from the microbubbles. These harmonic signals may be received by the ultrasound probe and may be separated from the linear tissue signals. Due to their size, the microbubble contrast agents are intravascular tracers that cannot leave the intravascular compartment. Thus, during ultrasound imaging, controlled ultrasound pulses may be transmitted that suppress tissue imaging while visualizing the microbubbles. Contrast-enhanced ultrasound imaging may then provide for direct visualization of certain anatomical features, such as liver lesions and intraplaque neovascularization, as the presence of microbubbles in plaque is indicative of an intraplaque neovessel. Microbubble distribution in an anatomical region of interest, such as the liver, artery walls, etc., may be evaluated to diagnose or rule out disease, monitor disease progression, etc. For example, a patient exhibiting atherosclerotic plaques may be evaluated over time to monitor progression of atherosclerosis disease. This evaluation may include quantifying the number, density, and/or distribution of microbubbles present in the plaque as a marker for disease progression.

Thus, when evaluating progression of a condition in a patient, a clinician may count the number of contrast agent microbubbles present in one or more anatomical regions. However, this process is time-consuming and may lead to inconsistent microbubble counts across different clinicians and different patients, and even across different imaging sessions of the same patient. In particular, if the microbubble count is determined over time for a patient to track atherosclerosis progression, inconsistent microbubble counts may lead to inaccurate determinations of disease progression, which could negatively impact patient care.

Thus, according to embodiments disclosed herein, microbubble density, distribution, and/or number within a target anatomical feature, such as a plaque, lesion, etc., may be determined automatically using an artificial intelligence-based model that is trained to segment the target anatomical feature in a contrast-enhanced image and generate a density map of the microbubbles in the segmented target anatomical feature. The automatically determined microbubble density map may be displayed on a display device and/or saved as part of a patient exam (e.g., in the patient's medical record). The density map may be similar to a heat map, with sub-regions of the density map having different microbubble densities within the target anatomical feature represented on the density map in different colors or shading. In doing so, contrast agent microbubble characterization may be more consistent across different patients and across different imaging sessions, which may improve patient care and reduce clinician workflow demands.

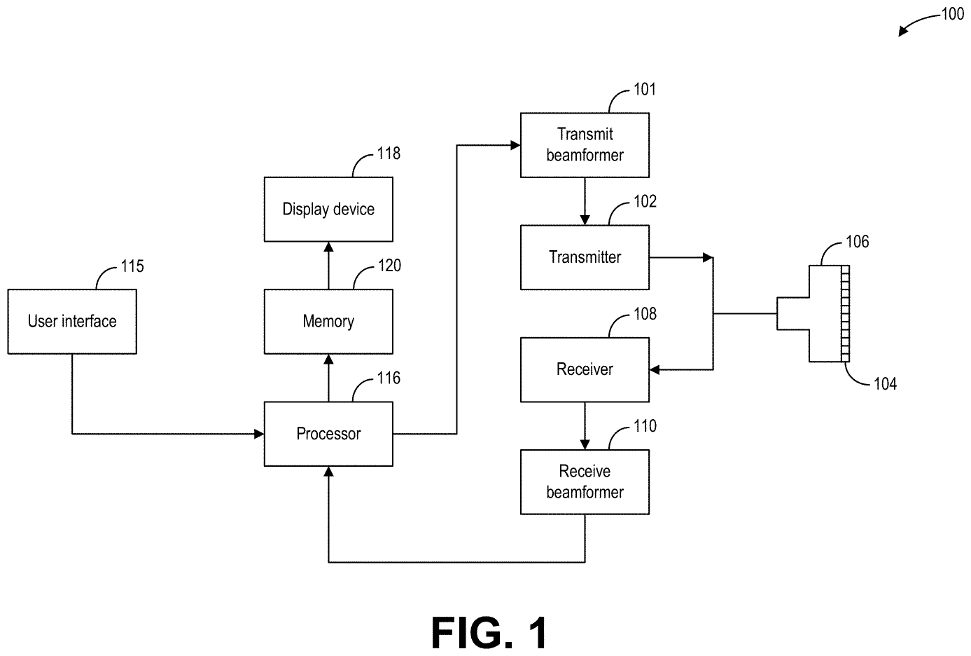

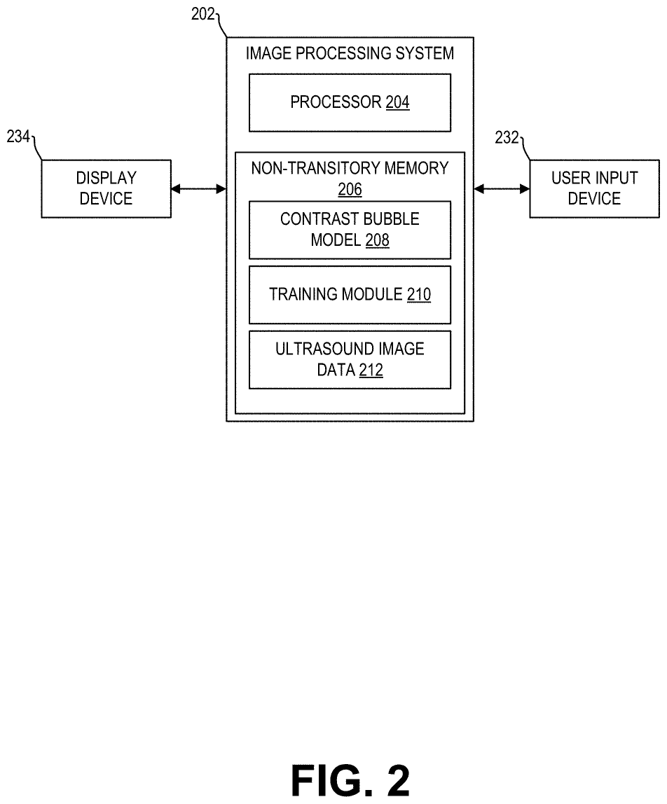

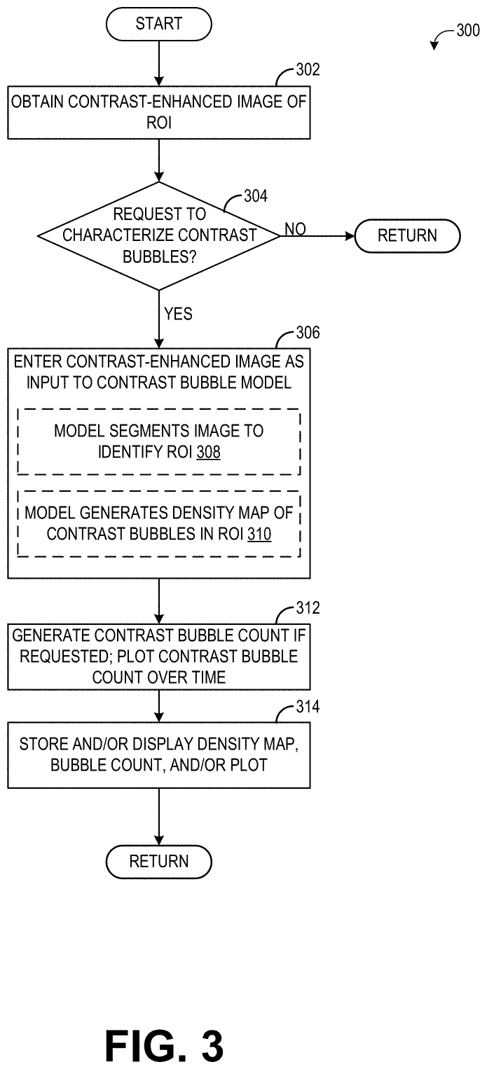

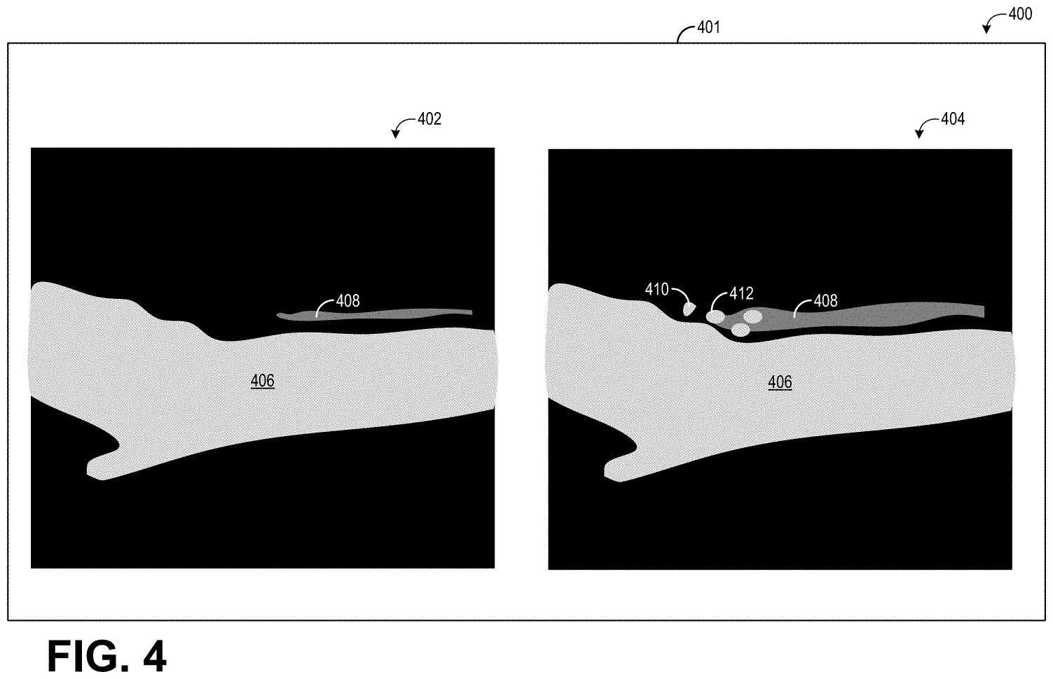

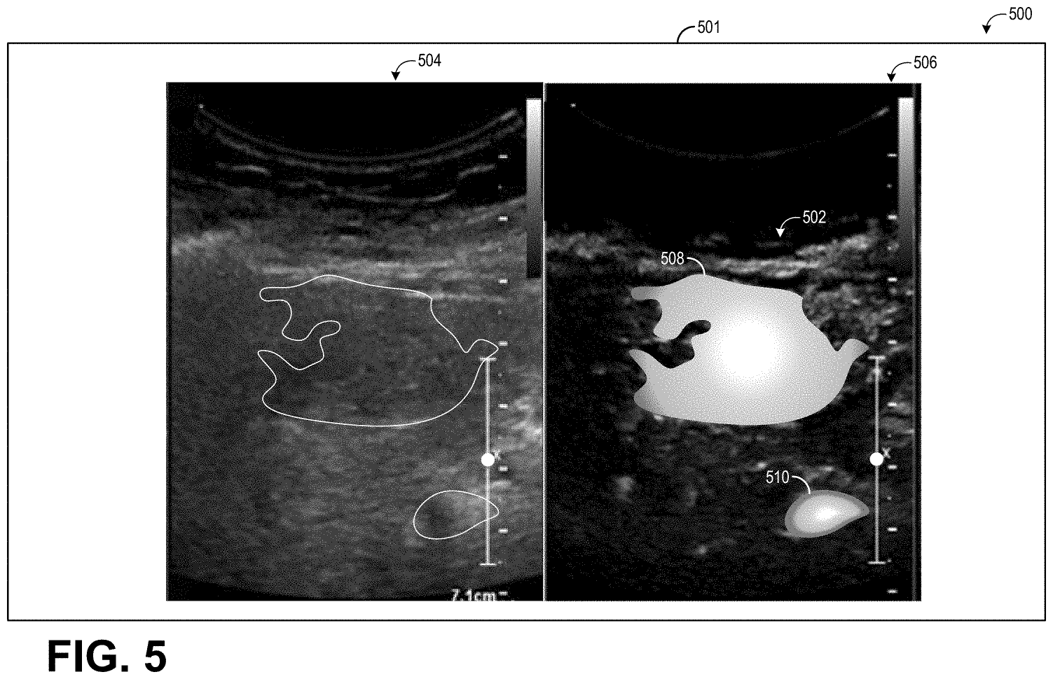

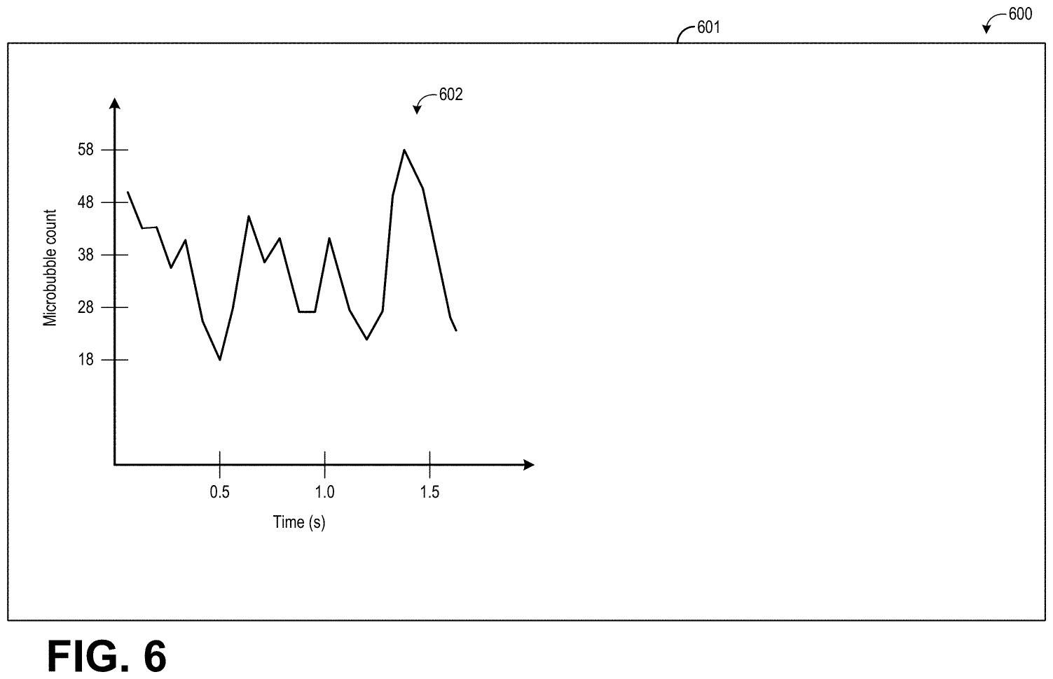

An ultrasound imaging system, such as the ultrasound imaging system of FIG. 1, may be used to obtain contrast-enhanced images, which may be entered as input to a contrast bubble model stored on an image processing system, such as the image processing system of FIG. 2. The contrast bubble model may be trained to segment a target region of interest (ROI) in a contrast-enhanced image and determine a number and/or density of contrast agent microbubbles in the target ROI, according to the method shown in FIG. 3. A visual representation of the number and/or density of microbubbles may be output for display on a display device, such as part of the graphical user interfaces shown in FIGS. 4 and 5. In some examples, a plot of microbubble count over time may be generated based on the density maps output by the contrast bubble model, as shown by FIG. 6.

Referring to FIG. 1, a schematic diagram of an ultrasound imaging system 100 in accordance with an embodiment of the disclosure is shown. The ultrasound imaging system 100 includes a transmit beamformer 101 and a transmitter 102 that drives elements (e.g., transducer elements) 104 within a transducer array, herein referred to as probe 106, to emit pulsed ultrasonic signals (referred to herein as transmit pulses) into a body (not shown). According to an embodiment, the probe 106 may be a one-dimensional transducer array probe. However, in some embodiments, the probe 106 may be a two-dimensional matrix transducer array probe. As explained further below, the transducer elements 104 may be comprised of a piezoelectric material. When a voltage is applied to a piezoelectric crystal, the crystal physically expands and contracts, emitting an ultrasonic spherical wave. In this way, transducer elements 104 may convert electronic transmit signals into acoustic transmit beams.

After the elements 104 of the probe 106 emit pulsed ultrasonic signals into a body (of a patient), the pulsed ultrasonic signals are back-scattered from structures within an interior of the body, like blood cells or muscular tissue, to produce echoes that return to the elements 104. The echoes are converted into electrical signals, or ultrasound data, by the elements 104 and the electrical signals are received by a receiver 108. The electrical signals representing the received echoes are passed through a receive beamformer 110 that outputs radio frequency (RF) data. Additionally, transducer element 104 may produce one or more ultrasonic pulses to form one or more transmit beams in accordance with the received echoes.

According to some embodiments, the probe 106 may contain electronic circuitry to do all or part of the transmit beamforming and/or the receive beamforming. For example, all or part of the transmit beamformer 101, the transmitter 102, the receiver 108, and the receive beamformer 110 may be situated within the probe 106. The terms “scan” or “scanning” may also be used in this disclosure to refer to acquiring data through the process of transmitting and receiving ultrasonic signals. The term “data” may be used in this disclosure to refer to either one or more datasets acquired with an ultrasound imaging system. In one embodiment, data acquired via ultrasound system 100 may be used to train a machine learning model. A user interface 115 may be used to control operation of the ultrasound imaging system 100, including to control the input of patient data (e.g., patient medical history), to change a scanning or display parameter, to initiate a probe repolarization sequence, and the like. The user interface 115 may include one or more of the following: a rotary element, a mouse, a keyboard, a trackball, hard keys linked to specific actions, soft keys that may be configured to control different functions, and a graphical user interface displayed on a display device 118.

The ultrasound imaging system 100 also includes a processor 116 to control the transmit beamformer 101, the transmitter 102, the receiver 108, and the receive beamformer 110. The processer 116 is in electronic communication (e.g., communicatively connected) with the probe 106. For purposes of this disclosure, the term “electronic communication” may be defined to include both wired and wireless communications. The processor 116 may control the probe 106 to acquire data according to instructions stored on a memory of the processor, and/or memory 120. The processor 116 controls which of the elements 104 are active and the shape of a beam emitted from the probe 106. The processor 116 is also in electronic communication with the display device 118, and the processor 116 may process the data (e.g., ultrasound data) into images for display on the display device 118. The processor 116 may include a central processor (CPU), according to an embodiment. According to other embodiments, the processor 116 may include other electronic components capable of carrying out processing functions, such as a digital signal processor, a field-programmable gate array (FPGA), or a graphic board. According to other embodiments, the processor 116 may include multiple electronic components capable of carrying out processing functions. For example, the processor 116 may include two or more electronic components selected from a list of electronic components including: a central processor, a digital signal processor, a field-programmable gate array, and a graphic board. According to another embodiment, the processor 116 may also include a complex demodulator (not shown) that demodulates the RF data and generates IQ data pairs representative of the echo signals. In another embodiment, the demodulation can be carried out earlier in the processing chain. The processor 116 is adapted to perform one or more processing operations according to a plurality of selectable ultrasound modalities on the data. In one example, the data may be processed in real-time during a scanning session as the echo signals are received by receiver 108 and transmitted to processor 116. For the purposes of this disclosure, the term “real-time” is defined to include a procedure that is performed without any intentional delay. For example, an embodiment may acquire images at a real-time rate of 7-20 frames/sec. The ultrasound imaging system 100 may acquire 2D data of one or more planes at a significantly faster rate. However, it should be understood that the real-time frame-rate may be dependent on the length of time that it takes to acquire each frame of data for display. Accordingly, when acquiring a relatively large amount of data, the real-time frame-rate may be slower. Thus, some embodiments may have real-time frame-rates that are considerably faster than 20 frames/sec while other embodiments may have real-time frame-rates slower than 7 frames/sec. The data may be stored temporarily in a buffer (not shown) during a scanning session and processed in less than real-time in a live or off-line operation. Some embodiments of the invention may include multiple processors (not shown) to handle the processing tasks that are handled by processor 116 according to the exemplary embodiment described hereinabove. For example, a first processor may be utilized to demodulate and decimate the RF signal while a second processor may be used to further process the data, for example by augmenting the data, prior to displaying an image. It should be appreciated that other embodiments may use a different arrangement of processors.

The ultrasound imaging system 100 may continuously acquire data at a frame-rate of, for example, 10 Hz to 30 Hz (e.g., 10 to 30 frames per second). Images generated from the data may be refreshed at a similar frame-rate on display device 118. Other embodiments may acquire and display data at different rates. For example, some embodiments may acquire data at a frame-rate of less than 10 Hz or greater than 30 Hz depending on the size of the frame and the intended application. A memory 120 is included for storing processed frames of acquired data. In an exemplary embodiment, the memory 120 is of sufficient capacity to store at least several seconds' worth of frames of ultrasound data. The frames of data are stored in a manner to facilitate retrieval thereof according to its order or time of acquisition. The memory 120 may comprise any known data storage medium.

In various embodiments of the present invention, data may be processed in different mode-related modules by the processor 116 (e.g., B-mode, Color Doppler, M-mode, Color M-mode, spectral Doppler, Elastography, TVI, strain, strain rate, and the like) to form 2D or 3D data. For example, one or more modules may generate B-mode, color Doppler, M-mode, color M-mode, spectral Doppler, Elastography, TVI, strain, strain rate, and combinations thereof, and the like. As one example, the one or more modules may process color Doppler data, which may include traditional color flow Doppler, power Doppler, HD flow, and the like. The image lines and/or frames are stored in memory and may include timing information indicating a time at which the image lines and/or frames were stored in memory. The modules may include, for example, a scan conversion module to perform scan conversion operations to convert the acquired images from beam space coordinates to display space coordinates. A video processor module may be provided that reads the acquired images from a memory and displays an image in real time while a procedure (e.g., ultrasound imaging) is being performed on a patient. The video processor module may include a separate image memory, and the ultrasound images may be written to the image memory in order to be read and displayed by display device 118.

In various embodiments of the present disclosure, one or more components of ultrasound imaging system 100 may be included in a portable, handheld ultrasound imaging device. For example, display device 118 and user interface 115 may be integrated into an exterior surface of the handheld ultrasound imaging device, which may further contain processor 116 and memory 120. Probe 106 may comprise a handheld probe in electronic communication with the handheld ultrasound imaging device to collect raw ultrasound data. Transmit beamformer 101, transmitter 102, receiver 108, and receive beamformer 110 may be included in the same or different portions of the ultrasound imaging system 100. For example, transmit beamformer 101, transmitter 102, receiver 108, and receive beamformer 110 may be included in the handheld ultrasound imaging device, the probe, and combinations thereof.

After performing a two-dimensional ultrasound scan, a block of data comprising scan lines and their samples is generated. After back-end filters are applied, a process known as scan conversion is performed to transform the two-dimensional data block into a displayable bitmap image with additional scan information such as depths, angles of each scan line, and so on. During scan conversion, an interpolation technique is applied to fill missing holes (i.e., pixels) in the resulting image. These missing pixels occur because each element of the two-dimensional block should typically cover many pixels in the resulting image. For example, in current ultrasound imaging systems, a bicubic interpolation is applied which leverages neighboring elements of the two-dimensional block. As a result, if the two-dimensional block is relatively small in comparison to the size of the bitmap image, the scan-converted image will include areas of poor or low resolution, especially for areas of greater depth.

Ultrasound images acquired by ultrasound imaging system 100 may be further processed. In some embodiments, ultrasound images produced by ultrasound imaging system 100 may be transmitted to an image processing system, where in some embodiments, the ultrasound images may be segmented by a machine learning model trained using ultrasound images and corresponding ground truth output. As used herein, ground truth output refers to an expected or “correct” output based on a given input into a machine learning model. For example, if a machine learning model is being trained to classify images of cats, the ground truth output for the model, when fed an image of a cat, is the label “cat”. In addition, the image processing system may further process the ultrasound images with one or more different machine learning models configured to count a number of contrast agent microbubbles based on the segmented ultrasound images.

Although described herein as separate systems, it will be appreciated that in some embodiments, ultrasound imaging system 100 includes an image processing system. In other embodiments, ultrasound imaging system 100 and the image processing system may comprise separate devices. In some embodiments, images produced by ultrasound imaging system 100 may be used as a training data set for training one or more machine learning models, wherein the machine learning models may be used to perform one or more steps of ultrasound image processing, as described below.

Referring to FIG. 2, image processing system 202 is shown, in accordance with an embodiment. In some embodiments, image processing system 202 is incorporated into the ultrasound imaging system 100. For example, the image processing system 202 may be provided in the ultrasound imaging system 100 as the processor 116 and memory 120. In some embodiments, at least a portion of image processing 202 is disposed at a device (e.g., edge device, server, etc.) communicably coupled to the ultrasound imaging system via wired and/or wireless connections. In some embodiments, at least a portion of image processing system 202 is disposed at a separate device (e.g., a workstation) which can receive images from the ultrasound imaging system or from a storage device which stores the images/data generated by the ultrasound imaging system. Image processing system 202 may be operably/communicatively coupled to a user input device 232 and a display device 234. The user input device 232 may comprise the user interface 115 of the ultrasound imaging system 100, while the display device 234 may comprise the display device 118 of the ultrasound imaging system 100, at least in some examples.

Image processing system 202 includes a processor 204 configured to execute machine readable instructions stored in non-transitory memory 206. Processor 204 may be single core or multi-core, and the programs executed thereon may be configured for parallel or distributed processing. In some embodiments, the processor 204 may optionally include individual components that are distributed throughout two or more devices, which may be remotely located and/or configured for coordinated processing. In some embodiments, one or more aspects of the processor 204 may be virtualized and executed by remotely-accessible networked computing devices configured in a cloud computing configuration.

Non-transitory memory 206 may store a contrast bubble model 208, training module 210, and ultrasound image data 212. Contrast bubble model 208 may include one or more machine learning models, such as deep learning networks, comprising a plurality of weights and biases, activation functions, loss functions, gradient descent algorithms, and instructions for implementing the one or more deep neural networks to process input ultrasound images. For example, contrast bubble model 208 may store instructions for implementing a segmentation model trained to identify and segment a target anatomical feature, such as an organ, a vessel, an artery, a lesion, etc., in a contrast-enhanced image. Contrast bubble model 208 may store further instructions for determining a number and/or density of contrast agent microbubbles in the target anatomical feature. The contrast bubble model 208 may include one or more neural networks. The contrast bubble model 208 may include trained and/or untrained neural networks and may further include training routines, or parameters (e.g., weights and biases), associated with one or more neural network models stored therein.

Thus, the contrast bubble model 208 described herein may be deployed to automatically determine the number and/or density of contrast agent microbubbles within an anatomical feature such as an artery wall, an organ (e.g., the liver), a lesion, etc. In some examples, the contrast bubble model 208 may include a U-net or other convolutional neural network architecture to segment a target anatomical feature in a contrast-enhanced image and/or a corresponding B-mode image (e.g., a non-contrast enhanced image taken in the same scan plane as the contrast-enhanced image) and may be trained using contrast-enhanced images and/or non-contrast enhanced images and/or cine loops where the target anatomical feature(s) have been annotated/identified by experts. The contrast bubble model 208 may further include another convolutional neural network (e.g., an auto-encoder model) trained to generate a density map of contrast agent microbubbles in the segmented anatomical feature. This network may be trained using contrast-enhanced images (and in some examples, also corresponding non-contrast images) of the anatomical feature, with the ground truth including a corresponding density map including defined sub-regions of the target anatomical feature and a density of contrast agent microbubbles in each sub-region, as determined via a bitmask and applied Gaussian filter. For the training, a user(s) may annotate images identifying, for one or more microbubbles in each image, the x and y position of each microbubble. As a result, a bitmask image with a non-zero value corresponding to marked microbubble positions is generated (e.g., with all other positions having a zero value). A convolution operation is performed with the bitmask using a normalized Gaussian filter to create the density map. The auto-encoder model may be trained with the contrast-enhanced images (as training input) and density maps (as ground truth output) to map a contrast image into a density map image. To produce a microbubble count, the density map may be integrated. Further still, in some examples, rather than using two separate networks to segment the target anatomical feature and characterize (e.g., determine the density and/or number) the microbubbles in the anatomical feature, the contrast bubble model 208 may include one network trained to both segment the target anatomical feature and characterize microbubbles in the target anatomical feature.

Non-transitory memory 206 may further include training module 210, which comprises instructions for training one or more of the machine learning models stored in the contrast bubble model 208. In some embodiments, the training module 210 is not disposed at the image processing system 202, and the contrast bubble model 208 thus includes trained and validated network(s).

Non-transitory memory 206 may further store ultrasound image data 212, such as ultrasound images captured by the ultrasound imaging system 100 of FIG. 1. The ultrasound image data 212 may include contrast-enhanced images and, at least in some examples, corresponding non-contrast enhanced images (e.g., non-contrast images of the same patient and at the same scan plane, for each contrast-enhanced image). Further, ultrasound image data 212 may store ultrasound images, ground truth output, iterations of machine learning model output, and other types of ultrasound image data that may be used to train the contrast bubble model 208, when training module 210 is stored in non-transitory memory 206. In some embodiments, ultrasound image data 212 may store ultrasound images and ground truth output in an ordered format, such that each ultrasound image is associated with one or more corresponding ground truth outputs. For example, ultrasound image data 212 may store sets of training data, where each set includes a contrast-enhanced image and a ground truth that includes a target anatomical feature annotated by an expert (e.g., a lesion annotated by a clinician) and/or a ground truth including a density map of contrast agent microbubbles in the anatomical feature as described above. Further, in examples where training module 210 is not disposed at the image processing system 202, the images/ground truth output usable for training the contrast bubble model 208 may be stored elsewhere.

In some embodiments, the non-transitory memory 206 may include components disposed at two or more devices, which may be remotely located and/or configured for coordinated processing. In some embodiments, one or more aspects of the non-transitory memory 206 may include remotely-accessible networked storage devices configured in a cloud computing configuration.

User input device 232 may comprise one or more of a touchscreen, a keyboard, a mouse, a trackpad, a motion sensing camera, or other device configured to enable a user to interact with and manipulate data within image processing system 202. In one example, user input device 232 may enable a user to make a selection of an ultrasound image to use in training a machine learning model, to indicate or label a position of an target anatomical feature in the ultrasound image data 212, or for further processing using a trained machine learning model.

Display device 234 may include one or more display devices utilizing virtually any type of technology. In some embodiments, display device 234 may comprise a computer monitor, and may display ultrasound images. Display device 234 may be combined with processor 204, non-transitory memory 206, and/or user input device 232 in a shared enclosure, or may be peripheral display devices and may comprise a monitor, touchscreen, projector, or other display device known in the art, which may enable a user to view ultrasound images produced by an ultrasound imaging system, and/or interact with various data stored in non-transitory memory 206.

It should be understood that image processing system 202 shown in FIG. 2 is for illustration, not for limitation. Another appropriate image processing system may include more, fewer, or different components.

FIG. 3 shows a flow chart illustrating an example method 300 for automatically characterizing contrast agent microbubbles in a contrast-enhanced ultrasound image of a region of interest (ROI), such as an artery, a lesion, an organ, etc., according to an embodiment. Method 300 is described with regard to the systems and components of FIGS. 1-2, though it should be appreciated that the method 300 may be implemented with other systems and components without departing from the scope of the present disclosure. Method 300 may be carried out according to instructions stored in non-transitory memory of a computing device, such as image processing system 202 of FIG. 2.

At 302, method 300 includes obtaining a contrast-enhanced image of an ROI.

Obtaining the contrast-enhanced image may include operating an ultrasound probe (e.g., probe 106 of FIG. 1) in a contrast-enhanced mode to image a patient who has been administered a bolus of an ultrasound contrast agent. The ultrasound contrast agent may include microbubbles of gas contained in a shell, such as octafluoropropane (perflutren) with an albumin shell or sulfur hexafluoride with a phospholipid shell. During contrast-enhanced imaging to obtain the contrast-enhanced image, the ultrasound probe may be controlled to emit pulses of ultrasonic energy having a low mechanical index, which may suppress tissue imaging, induce resonance behavior in the microbubbles, and prevent or reduce destruction of the microbubbles. However, the ultrasound probe may be controlled in a different manner without departing from the scope of this disclosure, such as at a higher mechanical index to cause destruction of the microbubbles. In some examples, the contrast-enhanced image may be a super-resolution contrast-enhanced image. The super-resolution contrast-enhanced image may be generated by using ultrafast plane wave imaging technology, using a deep learning convolutional neural network to map low-resolution contrast-enhanced ultrasound frames to a highly resolved contrast-enhanced frames, or using high frequency transducers. Super resolution contrast images have higher spatial resolution, which allows for more robust separation and identification of microbubbles. The contrast bubble model described herein may produce higher accuracy results using super resolution contrast images vs normal resolution images, but the microbubble density map generation and bubble count described herein may be performed on normal resolution images.

At 304, method 300 includes determining if a request to characterize contrast agent microbubbles has been received. The request to characterize the microbubbles may be received via user input. For example, an operator of the ultrasound imaging system may enter an input via a user input device (e.g., user interface 115 and/or user input device 232) requesting that the contrast agent microbubbles be characterized. In some examples, the user input requesting the contrast microbubbles be characterized may be received while the operator is actively imaging a patient, and thus the request may include a request to characterize the microbubbles using a particular image or series of images (e.g., a most currently acquired or stored contrast-enhanced image). In some examples, the request to characterize the microbubbles may be received from the ultrasound imaging system as part of an automated or semi-automated workflow.

If a request to characterize the microbubbles has not been received, method 300 returns. When no request is received to characterize the microbubbles, the ultrasound system may continue to acquire ultrasound images (whether contrast-enhanced, non-contrast enhanced, or in another imaging mode) when requested (e.g., when the ultrasound probe is powered on and in contact with an imaging subject), and may continue to assess if a request to characterize the contrast agent microbubbles is received.

If a request to characterize the microbubbles is received, method 300 proceeds to 306 to enter the contrast-enhanced image as input to a contrast bubble model. In some examples, the contrast-enhanced image that is obtained at 302 may be acquired by the ultrasound system in response to the request to characterize the contrast microbubbles. In other examples, the contrast-enhanced image may be obtained from memory. In some examples, the contrast-enhanced image that is entered into the model may be selected by a user, e.g., the operator of the ultrasound imaging system may select a contrast-enhanced image from a plurality of contrast-enhanced images stored in memory of the ultrasound imaging system, or the operator may indicate via user input that a currently-displayed contrast-enhanced image may be used for the microbubble characterization.

In some examples, the request to characterize the microbubbles may include an indication of which anatomical feature/ROI the contrast agent microbubbles are to be characterized (e.g., a request to characterize the microbubbles in an artery, in a lesion, in an organ, etc.). The contrast-enhanced image that is entered into the model (e.g., obtained at 302) may include the indicated anatomical feature/ROI.

The contrast bubble model (e.g., contrast bubble model 208) may include one or more deep learning/machine learning models trained to identify the ROI/anatomical feature of interest in the contrast-enhanced image and characterize the microbubbles in the ROI/anatomical feature. The contrast bubble model may perform image segmentation on the contrast-enhanced image and/or a corresponding non-contrast image (e.g., a B-mode image) to identify the borders of the ROI (e.g., the borders of a lesion in the contrast-enhanced image) and then characterize the microbubbles within the identified borders. Thus, as indicated at 308, the contrast bubble model may segment the contrast-enhanced image to identify and define the borders of the ROI. In some examples, a corresponding non-contrast image may be segmented to identify the borders of the ROI, and the borders of the ROI may be mapped/translated to the contrast-enhanced image (e.g., assuming the two images are of the same scan plane and region and that no or minimal patient or probe motion has occurred between acquisitions of the non-contrast enhanced image and contrast-enhanced image).

Characterizing the microbubbles may include generating a density map of microbubble density within the ROI, determining a number of microbubbles within the ROI (or within one or more sub-regions of the ROI) based on the density map, determining a change in microbubble density and/or number over time, or another characterization. As indicated at 310, upon entering the contrast-enhanced image to the contrast bubble model, the contrast bubble model may generate a density map of microbubbles in the ROI. The contrast bubble model may be trained to generate the density map of the microbubbles, which may include determining a density of the microbubbles in different sub-regions of the ROI. For example, the contrast-enhanced images may be super-resolution contrast-enhanced ultrasound images that allow a user to visualize contrast bubbles with high resolution. As explained above with respect to FIG. 2, to generate a density map, the bubble count model may be trained using a convolution operation with a Gaussian kernel (normalized) on a bitmask image (e.g., an image where a user has indicated at least one bubble location; at each bubble location, there will be a non-zero value). The bubble count model will then be trained to map the contrast-enhanced image to the density map, and the density map may be integrated to obtain the bubble count. Thus, once the contrast bubble model is deployed, the trained model utilizes the contrast image as an input and outputs the density map for the display and produces a micro-bubble count. The sub-regions may be predefined (e.g., a grid of squares of equal size) or the sub-regions may be defined by the contrast bubble model based on the distribution of microbubbles in the ROI (e.g., groups of pixels having the same or similar brightness may be defined as a sub-region). The density map may be in the style of a heat map, with each sub-region defined by a visual border and/or an indication of the number and/or density of the microbubbles in each sub-region. The indication of the number and/or density of the microbubbles in each sub-region may include numerals indicating the number and/or density of microbubbles, coloring or patterning of the sub-regions indicating the number and/or density, or another suitable visual representation of the number and/or density of each sub-region.

At 312, a contrast bubble count indicative of the number of contrast agent microbubbles in the entire ROI and/or each sub-region may be generated if requested. Further, if requested, a plot of contrast bubble count over time may be generated. As explained above, to determine the contrast bubble count, the density map may be integrated. The plot of contrast bubble count over time may be generated by determining the contrast bubble count for a plurality of images taken over time for the patient. The plot may show how the number of contrast bubbles changes over the course of contrast agent uptake and washout. A plot may be generated for the entire ROI (e.g., for the segmented ROI, or for a user-specified sub-region of the ROI).

At 314, the density map, bubble count, and/or bubble count plot may be stored in memory of the ultrasound imaging system and/or output for display on a display device (e.g., display device 118 or display device 234). In some examples, the density map may be displayed as an overlay on the contrast-enhanced image or on a B-mode image of the same target anatomical feature/region. Further, the density map, bubble count, and/or bubble count plot may be sent to a remote device, such as a device storing an electronic medical record database and/or a picture archiving and communication system (e.g., as part of a patient exam that includes ultrasound images of the patient). Method 300 then returns.

Thus, method 300 provides for automatically determining, via a contrast bubble model, a microbubble count of contrast agent microbubbles in a region of interest of a contrast-enhanced ultrasound image. The region of interest within the contrast-enhanced image may be determined automatically by the contrast bubble model. For example, the contrast bubble model may be trained to identify the region of interest in the contrast-enhanced image. The region of interest may be an anatomical feature such as a specific organ, a lesion, an artery wall, or other anatomical feature. However, in other examples, the region of interest may be defined by a user, such as the user entering a user input defining the border of the region of interest.

The contrast bubble model may be trained to determine the number of contrast agent microbubbles in the entirety of the region of interest. In some examples, the contrast bubble model may be trained to divide the region of interest into two or more sub-regions, and determine the number of contrast agent microbubbles in each sub-region. In some examples, the contrast bubble model may be trained to determine the density of the contrast agent microbubbles in each sub-region. The contrast bubble model may be trained to output a visual indication of the number of contrast agent microbubbles. In some examples, the visual indication may take the form of a density map.

FIG. 4 shows an example graphical user interface (GUI) 400 that may be displayed on a display device 401 (such as display device 118 and/or display device 234). GUI 400 may include two microbubble density maps as output by the contrast bubble model described herein, using two contrast-enhanced images as inputs. In each density map, sub-regions of the respective contrast-enhanced image are depicted in a color indicative of the density of microbubbles within that sub-region.

A first density map 402 is output by the contrast bubble model in response to a first contrast-enhanced image being input to the contrast bubble model. The first contrast-enhanced image may be an image of a carotid artery of a patient acquired upon administration of an ultrasound contrast agent, with an ultrasound probe controlled in a contrast mode (e.g., a low mechanical index). The first density map 402 represents the density of the contrast agent microbubbles as determined by the contrast bubble model, in two sub-regions. A first sub-region 406 represents the density of contrast agent microbubbles in the lumen of the carotid artery and a second sub-region 408 represents the density of contrast agent microbubbles in the wall of the carotid artery. The first sub-region 406 may be relatively bright, indicating a relatively high density of microbubbles. The second sub-region 408 may be less bright, indicating a lower density of microbubbles. Any remaining areas of the first density map 402 are black, indicating either no microbubbles were detected in those regions, or that those regions were not assessed by the contrast bubble model for the presence of microbubbles.

A second density map 404 is output by the contrast bubble model in response to a second contrast-enhanced image being input to the contrast bubble model. The second contrast-enhanced image may be an image of the carotid artery of the patient acquired approximately 10 seconds after acquisition of the first contrast-enhanced image. The second density map 404 represents the density of the contrast agent microbubbles as determined by the contrast bubble model, in the first and second sub-regions 406, 408, as well as additional sub-regions. Owing to the additional time following administration of the contrast agent when the second contrast-enhanced image was acquired, the microbubbles traveled to an atherosclerotic plaque, resulting in the second sub-region 408 growing in size and the inclusion of additional sub-regions having the relatively high density of microbubbles, such as third sub-region 410 and fourth sub-region 412. The density maps generated by the contrast bubble model, such as the second density map 404, may allow for diagnosis of atherosclerosis or other conditions (e.g., lesion neovascularization). Further, by monitoring density maps of the same patient over time, a clinician may track disease progress. For example, if the patient described above was imaged intermittently (e.g., every 3-6 months, every year) after initial diagnosis or suspicion of atherosclerosis, the progression of the atherosclerosis may be monitored based at least in part on the change in the density maps generated by the contrast bubble model, e.g., increasing number of sub-regions, increasing density of sub-regions, increasing size of sub-regions, etc.

FIG. 5 shows an example graphical user interface (GUI) 500 that may be displayed on a display device 501 (such as display device 118 and/or display device 234). GUI 500 includes a microbubble density map 502 as output by the contrast bubble model described herein, overlaid on the contrast-enhanced image 506 used as input to generate the density map. A B-mode image 504 is also displayed, including borders of two ROIs as segmented by the contrast bubble model. The B-mode image 504 may be an image of a liver of a patient acquired before administration of a contrast agent, and the contrast-enhanced image 506 may be an image of the liver acquired after administration of the contrast agent. The density map 502 includes two ROIs, a first ROI 508 and a second ROI 510, with the density of microbubbles within each ROI indicated by the colors and distribution of colors within that ROI (e.g., darker indicating lower density and lighter indicating higher density). Referring to the second ROI 510 as an example, the density of the second ROI 510 is not uniform, and the second ROI includes regions of different density, which may be referred to as sub-regions. The contrast bubble model may automatically determine the density in each sub-region and the location/distribution of the sub-regions. In some examples, the density in an ROI may be uniform. While gray-scale coloring is shown in FIG. 5, in some examples the density map may indicate different densities with other colors (e.g., green being lower density and red being higher density) or with patterns.

FIG. 6 shows an example graphical user interface (GUI) 600 that may be displayed on a display device 601 (such as display device 118 and/or display device 234). GUI 600 includes a microbubble plot 602 that depicts microbubble count for a ROI over time. In one example, the plot 602 may depict microbubble count over time for the second ROI 510 of FIG. 5. A plurality of contrast-enhanced images, similar to contrast-enhanced image 506 of FIG. 5, may be acquired at a suitable frame rate, such as 10 Hz. Each image may be entered into the contrast bubble model. A density map may be generated by the contrast bubble model for each image. Each density map (at the second ROI 510) may be integrated to generate a bubble count for the second ROI for each image. These bubble counts may be plotted as a function of the relative time that each image was acquired.

A technical effect of automatically determining the number of contrast agent microbubbles in a region of interest in a contrast-enhanced ultrasound image is reduced operator workflow and increased consistency of contrast agent microbubble counts across patients and imaging sessions. Another technical effect of automatically determining microbubble density and outputting a density map of the microbubble density is that a pattern of microbubble density and distribution may be used by a clinician to diagnose or rule out a disease or track disease progression.

An embodiment of a method includes generating, via a contrast bubble model, a density map of contrast agent microbubbles in a region of interest (ROI) of a contrast-enhanced ultrasound image; and displaying the density map on a display device. In a first example of the method, the method further includes identifying, via the contrast bubble model, the ROI of the contrast-enhanced ultrasound image. In a second example of the method, which optionally includes the first example, the density map includes an indication of density of the contrast agent microbubbles in two or more sub-regions of the ROI. In a third example of the method, which optionally includes one or both of the first and second examples, the density map includes, for each sub-region, a visual indication of the density of the contrast agent microbubbles for that sub-region. In a fourth example of the method, which optionally includes one or more or each of the first through third examples, each visual indication includes a color or pattern. In a fifth example of the method, which optionally includes one or more or each of the first through fourth examples, displaying the density map on the display device comprises displaying the density map as an overlay on the contrast-enhanced image. In a sixth example of the method, which optionally includes one or more or each of the first through fifth examples, the contrast bubble model is a neural network and wherein generating the density map includes inputting the contrast-enhanced image to the neural network. In a seventh example of the method, which optionally includes one or more or each of the first through sixth examples, the method further includes storing the density map in memory as part of a patient exam. In an eighth example of the method, which optionally includes one or more or each of the first through seventh examples, the method further includes determining a microbubble count based on the density map. In a ninth example of the method, which optionally includes one or more or each of the first through eighth examples, determining the microbubble count based on the density map comprises integrating the density map.

An embodiment of a system includes a display device; an ultrasound probe; a memory storing instructions; and a processor communicatively coupled to the memory and when executing the instructions, configured to: acquire, via the ultrasound probe, a contrast-enhanced image of a region of interest (ROI) of a patient; enter the contrast-enhanced image as an input to a contrast bubble model that is trained to output a density map of the ROI based on the contrast-enhanced image, the density map including a density of contrast agent microbubbles in one or more sub-regions of the ROI of the contrast-enhanced image; and output the density map for display on the display device. In a first example of the system, the contrast bubble model is trained to identify the ROI. In a second example of the system, which optionally includes the first example, the contrast bubble model is a neural network stored in the memory. In a third example of the system, which optionally includes one or both of the first and second examples, the contrast bubble model is trained with a plurality of training data sets, each training data set including a respective training contrast-enhanced image and a corresponding training density map of contrast agent microbubble density within the training contrast-enhanced image. In a fourth example of the system, which optionally includes one or more or each of the first through third examples, the corresponding training density map is generated by generating a bitmask from the training contrast-enhanced image and applying a Gaussian filter to the bitmask, wherein the training contrast-enhanced image includes annotations indicating a location of each of one or more contrast agent microbubbles in a ROI of the training contrast-enhanced image. In a fifth example of the system, which optionally includes one or more or each of the first through fourth examples, the density map is displayed as an overlay on the contrast-enhanced image.

An embodiment of a method for an ultrasound system includes receiving a request to determine a microbubble count of a region of interest (ROI) of a contrast-enhanced ultrasound image; upon receiving the request, entering the contrast-enhanced image as an input to a model trained to output a microbubble count based on the contrast-enhanced ultrasound image; and outputting the microbubble count for display on a display device. In a first example of the method, the model is trained to output a density map based on the contrast-enhanced ultrasound image, the density map including a density of contrast agent microbubbles in one or more sub-regions of the ROI of the contrast-enhanced ultrasound image, and wherein the microbubble count is determined based on the density map. In a second example of the method, which optionally includes the first example, the method further includes generating a plot of microbubble counts over time and outputting the plot for display on the display device. In a third example of the method, which optionally includes one or both of the first and second examples, generating the plot of microbubble counts comprises entering a plurality of contrast-enhanced ultrasound images each including the ROI acquired over time to the model in order to obtain a plurality of microbubble counts, and plotting the plurality of microbubble counts as a function of time.

When introducing elements of various embodiments of the present disclosure, the articles “a,” “an,” and “the” are intended to mean that there are one or more of the elements. The terms “first,” “second,” and the like, do not denote any order, quantity, or importance, but rather are used to distinguish one element from another. The terms “comprising,” “including,” and “having” are intended to be inclusive and mean that there may be additional elements other than the listed elements. As the terms “connected to,” “coupled to,” etc. are used herein, one object (e.g., a material, element, structure, member, etc.) can be connected to or coupled to another object regardless of whether the one object is directly connected or coupled to the other object or whether there are one or more intervening objects between the one object and the other object. In addition, it should be understood that references to “one embodiment” or “an embodiment” of the present disclosure are not intended to be interpreted as excluding the existence of additional embodiments that also incorporate the recited features.

In addition to any previously indicated modification, numerous other variations and alternative arrangements may be devised by those skilled in the art without departing from the spirit and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation and use may be made without departing from the principles and concepts set forth herein. Also, as used herein, the examples and embodiments, in all respects, are meant to be illustrative only and should not be construed to be limiting in any manner.

1. A method, comprising:
generating, via a contrast bubble model, a density map of contrast agent microbubbles in a region of interest (ROI) of a contrast-enhanced ultrasound image; and
displaying the density map on a display device.

2. The method of claim 1, further comprising identifying, via the contrast bubble model, the ROI of the contrast-enhanced ultrasound image.

3. The method of claim 1, wherein the density map includes an indication of density of the contrast agent microbubbles in two or more sub-regions of the ROI.

4. The method of claim 3, wherein the density map includes, for each sub-region, a visual indication of the density of the contrast agent microbubbles for that sub-region.

5. The method of claim 4, wherein each visual indication includes a color or pattern.

6. The method of claim 1, wherein displaying the density map on the display device comprises displaying the density map as an overlay on the contrast-enhanced image.

7. The method of claim 1, wherein the contrast bubble model is a neural network and wherein generating the density map includes inputting the contrast-enhanced image to the neural network.

8. The method of claim 1, further comprising storing the density map in memory as part of a patient exam.

9. The method of claim 1, further comprising determining a microbubble count based on the density map.

10. The method of claim 9, wherein determining the microbubble count based on the density map comprises integrating the density map.

11. A system, comprising:
a display device;
an ultrasound probe;
a memory storing instructions; and
a processor communicatively coupled to the memory and when executing the instructions, configured to:
acquire, via the ultrasound probe, a contrast-enhanced image of a region of interest (ROI) of a patient;
enter the contrast-enhanced image as an input to a contrast bubble model that is trained to output a density map of the ROI based on the contrast-enhanced image, the density map including a density of contrast agent microbubbles in one or more sub-regions of the ROI of the contrast-enhanced image; and
output the density map for display on the display device.

12. The system of claim 11, wherein the contrast bubble model is trained to identify the ROI.

13. The system of claim 11, wherein the contrast bubble model is a neural network stored in the memory.

14. The system of claim 13, wherein the contrast bubble model is trained with a plurality of training data sets, each training data set including a respective training contrast-enhanced image and a corresponding training density map of contrast agent microbubble density within the training contrast-enhanced image.

15. The system of claim 14, wherein the corresponding training density map is generated by generating a bitmask from the training contrast-enhanced image and applying a Gaussian filter to the bitmask, wherein the training contrast-enhanced image includes annotations indicating a location of each of one or more contrast agent microbubbles in a ROI of the training contrast-enhanced image.

16. The system of claim 11, wherein the density map is displayed as an overlay on the contrast-enhanced image.

17. A method for an ultrasound system, comprising:
receiving a request to determine a microbubble count of a region of interest (ROI) of a contrast-enhanced ultrasound image;
upon receiving the request, entering the contrast-enhanced image as an input to a model trained to output a microbubble count based on the contrast-enhanced ultrasound image; and
outputting the microbubble count for display on a display device.

18. The method of claim 17, wherein the model is trained to output a density map based on the contrast-enhanced ultrasound image, the density map including a density of contrast agent microbubbles in one or more sub-regions of the ROI of the contrast-enhanced ultrasound image, and wherein the microbubble count is determined based on the density map.

19. The method of claim 17, further comprising generating a plot of microbubble counts over time and outputting the plot for display on the display device.

20. The method of claim 19, wherein generating the plot of microbubble counts comprises entering a plurality of contrast-enhanced ultrasound images each including the ROI acquired over time to the model in order to obtain a plurality of microbubble counts, and plotting the plurality of microbubble counts as a function of time.
